(19) United States
(12) Patent Application Publication
(10) Pub. No.: US 2020/0323935 A1
(43) Pub. Date: Oct. 15, 2020

(54) NOVEL METHOD FOR THE TREATMENT OF DYSPEPSIA, FOOD AND MEDICINE INTOLERANCES, IRRITABLE BOWEL SYNDROME AND OTHER FUNCTIONAL GASTROINTESTINAL CONDITIONS

(71) Applicant: G.I. Pharmaceuticals, Inc. dba E2BIO CONSULTANTS, INC., Skokie, IL (US)

(72) Inventors: Eli D. Ehrenpreis, M.D., Skokie, IL (US); Benjamin Ehrenpreis, Skokie, IL (US)

(73) Assignee: G.I. Pharmaceuticals, Inc. dba E2BIO CONSULTANTS, INC., Skokie, IL (US)

(21) Appl. No.: 16/830,334

(22) Filed: Mar. 26, 2020

Related U.S. Application Data
(60) Provisional application No. 62/831,757, filed on Apr. 10, 2019.

(51) Int. Cl. A61K 36/185; A61K 9/16; A61K 38/16

(52) U.S. Cl. CPC A61K 36/185; A61K 38/168; A61K 9/167

(57) ABSTRACT

Disclosed are methods for the treatment of functional gastrointestinal disorders. The invention involves using proteins that bind to sweet taste receptors in the gastrointestinal tract to modulate noxious symptoms that occur in patients. The methods involve the use of dried whole miracle berry, dried miracle berry juice, miracle berry extract and/or naturally derived or synthetic miraculin protein that is administered in a delayed release capsule or tablet. Use of a delayed release drug delivery system allows miraculin protein, the active ingredient of miracle berry, to bypass destruction in the acid environment of the stomach. This method also allows for the release of larger concentrations of the active ingredient(s) to be applied directly to portions of the gastrointestinal tract that contain specific sweet taste receptors. The method also suggests that other proteins that bind to sweet taste receptors may be used treat these functional gastrointestinal disorders.

### CROSS REFERENCE TO RELATED INVENTIONS

The present invention claims priority to U.S. Provisional Application 62/831,757 filed Apr. 10, 2019, which is further incorporated in its entirety by reference.

### BACKGROUND OF THE INVENTION

Patients with functional gastrointestinal (GI) disorders suffer from a variety of chronic symptoms related to abnormal digestion and bowel activities in the absence of organic disease. Included among these symptoms are abdominal pain and discomfort, abnormal bowel habits and abdominal bloating and dyspepsia. The symptom of complex occurring in patients with chronic dyspepsia is characterized by excessive fullness of the stomach, nausea, gastroesophageal reflux, early satiety and abdominal pain. Functional dyspepsia is diagnosed in patients with typical upper GI symptoms and a normal upper endoscopy. Patients with food intolerances develop upper and/or lower GI symptoms that are triggered by a variety of specific foods. Food intolerance is different from food allergies, which are organic conditions due to abnormal histamine release following exposure to an allergen. The causes of food allergies are poorly understood. Unexplained gastrointestinal symptoms related to intolerance of medications is another common problem that has been poorly characterized. Irritable bowel syndrome (IBS) is characterized by chronic abdominal pain or discomfort, diarrhea, constipation or both, abdominal distention and incomplete evacuation of the stool. As with other functional GI disorders, the causes and optimal treatments of IBS have not been fully elucidated.

Physiologic alterations in the enteric nervous system (ENS) are thought to play an important role in the development of functional GI disorders. The ENS is a highly complex arrangement of intrinsic and extrinsic neurons that integrate sensory information and motor function of the GI tract, the spinal cord, and the brain. Altered signaling between these systems appears to be a major factor in the augmentation and perpetuation of symptoms in patients with functional GI disorders. Within the ENS, signaling from the GI tract occurs in response to a variety of local stimuli. GI sensory input undergoes modulation in the spinal cord prior to transmission of information to the brain. Motor function of the GI tract is influenced by pathways from both the brain and the spinal cord. Thus, integrated signaling occurs between the gut and the brain; these connected activities are influenced by a variety of factors. Increased intestinal motility and mucosal hyperemia induced by stressful or painful experiences are examples of alteration of gut function induced by the brain and spinal cord.

The major intrinsic sensory neurons of the GI tract are located in groupings or plexi that are found beneath the mucosa (submucosal plexus) and between the muscle layers of the GI organs (muscular plexus). Diverse forms of sensory receptors are present within these GI tract nerve plexi. These receptors react to mechanical, thermal, osmotic and chemical stimuli.

Receptors that react to the chemical environment of the GI tract are known as chemoreceptors. Several studies have shown that there are chemoreceptors in the GI tract that relay information about the “taste” of the GI contents. Just like other intestinal receptors, information from the GI chemoreceptors for “taste” is believed to produce activities in the enteric nervous system. It has been hypothesized that these receptors may play a role in inducing intestinal reactions after ingestions of toxic chemicals and for regulation of glucose absorption. However, the inventors are the first to suggest that these receptors have a role in the pathophysiology of functional GI disorders. In addition, this invention represents a novel method to treat these disorders by altering sweet taste receptors in the GI tract.

Functional dyspepsia is highly prevalent in the United States and appears to occur in between 20-30% of the population. During their lifetime, about 10% of people will develop some form of food intolerance. Functional abdominal pain lasting more than 8 weeks occurs up to 24% of children and is associated with a disruption of school and family life. Irritable bowel syndrome is the most common functional GI disorder and affects up to 15% of the world's population. IBS results in 2.4 and 3.5 million physician appointments annually in the United States. Aside from costs of care, the economic consequences of functional bowel disorders include decreased quality of life, and absenteeism from work. For example, the direct and indirect costs for IBS are more than $21 billion per year in the United States.

The present state of treating dyspepsia, medication and food intolerances includes dietary adjustment, stress reduction, relaxation techniques, hypnosis, acupuncture and probiotic therapy. Pharmacotherapies include acid blocking drugs and antidepressants. These pharmacotherapies have potentially significant side effects. Some medications for the treatment of functional GI disorders directly alter intestinal receptors; several of these have been or restrict removed in the pharmaceutical market following the identification of serious and potentially life-threatening side effects during post-marketing surveillance.

The present state of treating IBS is like functional dyspepsia (dietary adjustment, stress reduction, relaxation techniques, hypnosis, acupuncture and probiotic therapy). Pharmacotherapies include antispasmodic drugs and antidepressants. These pharmacotherapies have potentially significant side effects. Newer non absorbable antibiotics have become a mainstay of treatment for diarrhea-predominant IBS, but these are indicated for short-term use. A variety of medications have been developed for IBS that interact alter intestinal receptors; several of these have subsequent removal or restriction from the pharmaceutical market because of serious and potentially life-threatening side effects during post-marketing surveillance. Newer treatments that affect locally acting inhibitors of the sodium/hydrogen exchanger 3 (NHE3), prosecretory agonists of intestinal chloride channels and newer drugs that alter serotonin 5HT receptors are also being used to treat IBS

Although some patients with dyspepsia and other functional GI disorders respond well to currently available treatments, many if these patients have refractory symptoms. For example, a recent study revealed that 40% of IBS patients are dissatisfied with their current medical treatment and 62% assume that they would not find a satisfactory method to manage their symptoms. Hence, a need remains for safe and effective treatments for functional bowel disorders.

The present invention represents the use of a completely new method for treating functional GI disorders. The inventors discovered this method after swallowing several berries from the miracle fruit plant, Synsepalum dulcificum and noticing a reduction of gastrointestinal symptoms after consuming a large, spicy meal. They have subsequently studied the effects of whole miracle berry, miracle berry extract and purified miraculin peptide for use in preventing noxious gastrointestinal symptoms from eating foods that produce symptoms of dyspepsia.

Miracle berries are normally chewed to release miraculin, a glycoprotein that binds to the sweet taste receptors, hT1R2-hT1R3, in the mouth. Following binding to these receptors that are primarily located on the tongue, miraculin peptide directly activates hT1R2 and hT1R3. In the mouth, G-protein coupling within these receptors produces a perception of sweet taste. Because of this property of miraculin peptide, chewing miracle fruit berries followed by the consumption of bitter substances such as lemon and apple cider vinegar changes the perceived taste of these substances from bitter to sweet. Based on the aforementioned mechanism, a few small clinical studies have determined that chewing miracle berries may be useful in the management of taste disturbances that occur in patients undergoing chemotherapy and radiation therapy for cancer. Miracle berry has also become popularized, with chewing of miracle berries combined followed by putting sour substances such as lemons and vinegar in the mouth for “taste parties”.

The presently described method, using the whole fruit of Synsepalum dulcificum, extracts of the fruit of Synsepalum dulcificum, miraculin protein as well as other peptides and proteins that bind to taste receptors in the small intestine and large intestine, represents a new pathway in the management of functional gastrointestinal disorders.

Curculin is a naturally sweet protein found in the fruit of Curculigo latifolia. Like miraculin protein, curculin is a taste modifying protein that induces a sweet taste when combined with taste receptors in the mouth. Several other naturally derived proteins that alter sweet taste receptors have been identified. These include thaumatin, monellin, mabinlin, and pentadin. Thaumatin is a protein from the katemfe fruit (Thaumatococcus daniellii Bennett) that is several thousand times sweeter than commonly consumed sugar. Monellin is a protein derived from the serendipity berry (Dioscoreophyllum cumminsii) that is approximately 3000 times sweeter than sucrose. Mabinlin is a protein that is derived from the fruit of Capparis masaikai. Mabinlin is 400 times sweeter than sucrose. Pentadin, is a protein that is derived from the fruit of Pentadiplandra brazzeana and is 500 times sweeter than sucrose. These substances have the potential to not only affect sweet taste receptors in the mouth, but also to alter gastrointestinal receptors. Therefore, this invention represents the method of including any or all the aforementioned taste altering proteins as treatments for functional gastrointestinal disorders.

### SUMMARY OF THE INVENTION

A first aspect of the present invention is directed to a method of treating functional gastrointestinal conditions, comprising the use of naturally occurring or synthetic proteins that binds to taste receptors that are present on the mucosa of the GI tract of a patient in need thereof with a therapeutically effective agent. This treatment is administered in a delayed-release form to prevent its destruction in the stomach and to bind most specifically to taste receptors in the small and large intestine. Altering the function of taste receptors in the gastrointestinal tract represents a novel approach to the management of a variety of functional bowel disorders, including dyspepsia, food intolerances, gastrointestinal side effects of some medications and IBS.

The method involves administration of a pharmacologic substances (natural or synthetic) that bind to and alter gastrointestinal chemoreceptors for taste, specifically those receptors that are present in the small intestine and the large intestine. Heretofore, gastrointestinal taste receptors have not been considered to play a role in the pathogenesis or treatment of functional gastrointestinal disorders including dyspepsia, food and medication intolerances, IBS or various other conditions affecting the gastrointestinal tract. It is known in the literature that altered sensory pathways of the enteric nervous system are a key component of the symptoms experienced by patients with these disorders. Prior to this described method, activators and inhibitors of cholinergic, serotonergic, adrenergic, histaminergic, dopaminergic, gastrineric, nitric oxide, proton pumps and chloride transport receptors have been the focus of attention for pharmacologic intervention in functional GI disorders.

The primary substances of interest are whole berries from the miracle berry plant, Synsepalum dulcificum, miracle berry extract and naturally derived and synthetic miraculin protein, all administered in delayed release forms. The method may also be applied to other taste modifying natural proteins, including curculin, thaumatin, monellin, mabinlin, and pentadin.

The inventive methods may be used to either treat ongoing symptoms or prevent the occurrence of these symptoms. Accordingly, one aspect of the present invention is directed to a method of ingesting a substance that interacts with gastrointestinal taste receptors on a regular basis, such as three to four times daily, particularly before meals or snacks. Alternatively, the treatment may be used as needed prior to ingestion of a meal that is known to initiate the symptoms in a patient with a functional GI disorder. In this way, the treatment works by inhibiting, delaying the onset, or reducing the severity of the symptoms of functional gastrointestinal symptoms. In this setting, a prophylactically effective amount of the treatment interacts with gastrointestinal taste receptors prior to activation of these receptors by food or nutrient-derived stimulants.

Another aspect of the present invention is directed to a method of ingesting a substance that interacts with gastrointestinal taste receptors to lessen the severity of symptoms in a patient with a functional GI disorder. In this way, the treatment works by directly reducing the severity of the symptoms as they occur in a patient with a functional GI disorder. In this setting, a therapeutically effective amount of the treatment interacts with gastrointestinal taste receptors to alter their activity and decrease the noxious stimuli that fuel the functional GI disorder.

In some embodiments of these therapeutic and prophylactic methods, the taste receptor activator agent may be administered in a delayed-release capsule that is specifically designed to remain intact while the active agent is in the stomach and to open and release the active agent after entering the small intestine or colon. This method of administration of miracle berries, miracle, miracle berry extract, natural and synthetic miraculin protein or other types of proteins that interact with taste receptors has not been previously described. Because miraculin is a glycoprotein and other active substances utilized in this method are proteins, delaying their release until after these substances exit from the stomach prevents their destruction in the highly acidic environment (low pH of 0-3) of the stomach. In these embodiments, the agent may be disposed within a capsule that resists dissolution by acid in the stomach, and instead dissolves in the less acidic, neutral or basic (higher pH of 5-8) environment of the small intestine and the large intestine. This allows the agent to remain intact as the capsule travels within the stomach and is propelled in the small and large intestine. Once the capsule reaches the appropriate pH, the capsule dissolves and its contents are released. The released contents of the capsule then bind to the receptors to which they come into contact. This form of capsule is termed “pH dependent”.

In some embodiments of this method, the active agent is contained within a delayed-release capsule that dissolves slowly as it travels through the GI tract. This form of capsule allows for small amounts of the active ingredient to be released in all parts of the GI tract. Using this form of capsule, most of the active ingredient is still released outside (distal) to the stomach and remains safe from destruction within the stomach. On the other hand, some of the contents of the capsule's content have the potential to remain intact within the stomach and can interact with taste receptors in the lining of the stomach. This has the potential for treating functional gastrointestinal disorders of the including functional dyspepsia, food and medication intolerances and irritable bowel syndrome, since some of these noxious stimuli that produce dyspepsia, result from stimulation of sensory receptors located in the lining of the stomach.

Some forms of delayed release capsules are called “timed released” capsules, in which the active ingredient is mixed with a substance that slows the deliver of the substance once it enters the GI tract.

In some embodiments of these therapeutic and prophylactic methods, the taste receptor activating agent may be administered in a delayed-release tablet. In this embodiment, the tablet is enteric-coated. This results in a delay in the release of the taste receptor altering substance until the tablet has passed through the stomach to prevent the drug from being destroyed within the acidic environment of the stomach or inactivated by gastric juice.

In some embodiments of these therapeutic and prophylactic methods, the taste receptor activating agent may be administered in an extended-release tablet. These extended release tablets are designed to allow the taste receptor-altering substances to be exposed to the entire gastrointestinal tract for a prolonged period after ingestion.

Active components of the treatment may also be assembled in microgranules that are resistant to enzymatic and acid destruction. These microgranules may then be incorporated into PH-dependent, timed release capsules or delayed-release or extended-release tablets.

In some embodiments of these therapeutic and prophylactic methods, the taste receptor-altering substance is powdered miracle berry in a dried form that is placed with a delayed-release, or an extended release capsule or tablet. The contents of each capsule will range from 1-10 dried miracle berries and the total dose for each usage will range from 1-5 capsules.

In some embodiments of these therapeutic and prophylactic methods, the taste receptor-altering substance is the juice of the miracle berry which has been rendered into a dried form that is placed with a delayed-release, or an extended release capsule or tablet. The contents of each capsule will range from the juice of 1-30 miracle berries and the total dose for each usage will range from 1-5 capsules.

In some embodiments of these therapeutic and prophylactic methods, the taste receptor-altering substance is an extract of the miracle berry which has been rendered into a dried form that is placed with a delayed-release, or an extended release capsule or tablet. The contents of each capsule will range from the extract from 1-30 miracle berries and the total dose for each usage will range from 1-5 capsules.

In some embodiments of these therapeutic and prophylactic methods, the taste receptor-altering substance is a commercially available form of miracle berry tablet which has been rendered and repurposed into a newly created form of delayed-release, or an extended release capsule or tablet. The contents of each capsule will range from 1-10 commercially prepared miracle berry tablets and the total dose for each usage will range from 1-5 capsules.

In some embodiments of these therapeutic and prophylactic methods, the taste receptor-altering substance a pharmaceutical grade of naturally occurring or synthetic miraculin protein which has been placed with a delayed-release, or an extended release capsule or tablet. The contents of each capsule will range from 0.001 to 1 mg of miraculin protein and the total dose for each usage will range from 1-5 capsules.

In some embodiments of these therapeutic and prophylactic methods, the taste receptor-altering substance is administered in a targeted delivery system consisting of delayed release capsules containing coated microspheres of miracle berry extract. The contents of each capsule will range from the extract from 1-30 miracle berries and the total dose for each usage will range from 1-5 capsules.

In some embodiments of these therapeutic and prophylactic methods, the taste receptor-altering substance is dried fruit from the Curculigo latifolia plant.

In some embodiments of these therapeutic and prophylactic methods, the taste receptor-altering substance is a commercially available form of Curculigo latifolia fruit tablet which has been rendered and repurposed into a newly created form of delayed-release, or an extended release capsule or tablet.

In some embodiments of these therapeutic and prophylactic methods, the taste receptor-altering substance is pharmaceutical grade curculin.

In some embodiments of these therapeutic and prophylactic methods, the taste receptor-altering substance is pharmaceutical grade natural or synthetic thaumatin, monellin, mabinlin, and pentadin or their parent fruit in a form that is placed with a delayed-release, or an extended release capsule or tablet.

In some embodiments of these therapeutic and prophylactic methods, the taste receptor-altering substance is the whole or dried forms of katemfe fruit containing thaumatin, serendipity berry containing monellin, the fruit of Capparis masaikai containing mabinlin, and the fruit of Pentadiplandra brazzeana that contains pentadin. These whole or dried fruits will be delivered to the small and/or large intestines with a delayed-release, or an extended release capsule or tablet.

In some embodiments of these therapeutic and prophylactic methods, the taste receptor-altering substance is extracts of katemfe fruit containing thaumatin, serendipity berry containing monellin, the fruit of Capparis masaikai containing mabinlin, and the fruit of Pentadiplandra brazzeana that contains pentadin. These fruit extracts will be delivered to the small and/or large intestines with a delayed-release, or an extended release capsule or tablet.

Without intending to be bound by theory, the present inventor believes that miracle berry, miracle berry juice, miracle berry extract and naturally occurring and synthetic miraculin peptide can be administered in a form that allows it to be exposed to taste receptors in the stomach, small intestine and large intestine. Alteration of these receptors by miracle berry, miracle berry juice, miracle berry extract and naturally occurring and synthetic miraculin peptide will result in the reduction of abdominal symptoms occurring from functional gastrointestinal disorders and food intolerances. The inventors have now tested this hypothesis by the administration of miracle berry extract in a delayed release capsule form that was produced by a compounding pharmacy, and purified miraculin administered in a delayed-release capsule. Two subjects performed a baseline study by ingesting a meal that was known to cause abdominal symptoms (falafel sandwich) and recorded the gastrointestinal effects of these meals over time using a scoring system based on a visual analogue scale. In subsequent studies, prior to ingesting this meal, the subjects ingested delayed release capsules containing miracle berry extract and purified miraculin protein in delayed release capsules and again recorded the gastrointestinal effects of the meals (See Tables 1-5).

As can be seen from the tables, miracle berry extract and purified miraculin markedly reduced gastrointestinal symptoms from the poorly tolerated meal in both subjects. These data confirm the hypothesis that miracle berry extract and purified miraculin administered in a delayed release form represent novel methods to limit or prevent noxious stimuli in the intestine and to decrease GI symptoms induced by poorly tolerated dietary substances, such as high fat content and spicy foods. This new method represents an innovative and previously undiscovered approach to treating functional gastrointestinal disorders.

### BRIEF DESCRIPTION OF THE TABLES

Tables 1-7 show the results of preliminary studies to test the efficacy of the described invention in two subjects with severe intolerance consumption of a meal containing falafel, a Middle Eastern food that contains fried chickpeas. In both subjects, consumption of a falafel meal caused severe symptoms of dyspepsia. In these studies, delayed release capsules containing miracle extract were used in the first part of the study. Each of these delayed release capsules contained the equivalent of juice from three miracle berries. The delayed release capsules containing the miracle berry extract were made in a compounding pharmacy by blending the ingredients with cellulose derivatives and refilling the capsules, thus producing a hydrophilic gel system.

Studies of miraculin protein were performed using purified miraculin that was placed in capsules that were made in a compounding pharmacy by blending the ingredients with cellulose derivatives and refilling the capsules, thus producing a hydrophilic gel system. Each purified miraculin capsule contained 0.2 mg of miraculin protein.

Both subjects consumed a baseline falafel meal in their first study. The first subject underwent two additional studies, one with two capsules of delayed release miracle berry extract consumed 30 minutes before a falafel meal. In the third study, the subject consumed 2 capsules, each containing 0.2 mg purified miraculin (total miraculin dose of 0.4 mg) in delayed release capsules 30 minutes before the meal.

The second subject performed a second study consuming a falafel meal 15 minutes after taking 2 capsules of miracle berry extract in delayed release capsules. In the third study, the second subjects subject took 1 capsule of 0.2 mg purified miraculin in a delayed release capsule 15 minutes before consumption of a falafel meal. In the fourth study, the second subject took two capsules of 0.2 mg purified miraculin (total dose of 0.4 mg), 15 minutes before a falafel meal. Subjects answered a simple questionnaire every 15 minutes for two hours. The questionnaire consisted of rating from 0-10 for the development of symptoms of their reaction to foods causing gastric distress, including the following: Abdominal Discomfort, Bloating, Gas Retention, Metallic Taste in the Mouth, Reflux and Diarrhea. A Global score was incorporated into the studies. In both subjects, a significant decrease in symptoms caused by the falafel meal was seen with both the miracle berry extract and the purified miraculin in delayed release capsules were ingested. Of note, the second subject had an even better result of improvement of dyspeptic symptoms after the use of two 0.2 mg capsules of miraculin, compared to one 0.2 mg capsule of miraculin.

Table 1-Study of dyspeptic symptoms after a falafel meal. Study consisted of the meal alone.

Results are Shown for Subject 1

Subject 1--Falafel Alone
Symptom Severity (0-10)

| TIME | Abdominal | | Gas | Metallic | | |
|---|---|---|---|---|---|---|
| (minutes) | discomfort | Bloating | retention | Taste | Reflux | Diarrhea |
| 0 | 0 | 0 | 0 | 0 | 3 | 0 |
| 15 | 1 | 2 | 2 | 0 | 3 | 0 |
| 30 | 2 | 4 | 0 | 0 | 4 | 0 |
| 45 | 1 | 4 | 0 | 0 | 5 | 0 |
| 60 | 1 | 4 | 1 | 0 | 4 | 0 |
| 75 | 2 | 3 | 2 | 0 | 0 | 0 |
| 90 | 2 | 1 | 2 | 0 | 1 | 0 |
| 105 | 2 | 2 | 2 | 4 | 5 | 5 |
| 120 | 4 | 3 | 4 | 1 | 6 | 0 |

Additional notes: Between hour 2 and 3, the subject described “feeling really bad with severe reflux, stomach churning. Reflux up to 7 or 8, bloating bad”. The subject ingested gastric acid blocking medications with gradual improvement of symptoms.

Table 2—Study of dyspeptic subject, two slow release capsules of dried miracle berry extract were ingested 30 minutes before the meal was consumed.

Results are Shown for Subject 1

Subject 1--Falafel plus Two Miracle Berry Extract Capsules
Symptom Severity (0-10)

| TIME | Abdominal | | Gas | Metallic | | |
|---|---|---|---|---|---|---|
| (minutes) | discomfort | Bloating | retention | taste | Reflux | Diarrhea |
| 0 | 0 | 0 | 0 | 0 | 0 | 0 |
| 15 | 0 | 0 | 0 | 0 | 0 | 0 |
| 30 | 0 | 0 | 0 | 0 | 0 | 0 |
| 45 | 0 | 0 | 0 | 0 | 0 | 0 |
| 60 | 0 | 0 | 0 | 0 | 0 | 0 |
| 75 | 0 | 0 | 0 | 0 | 0 | 0 |
| 90 | 2 | 2 | 0 | 0 | 0 | 0 |
| 105 | 1 | 0 | 0 | 0 | 0 | 0 |
| 120 | 0 | 0 | 0 | 0 | 0 | 0 |

Table 3—Study of a dyspeptic subject, two slow release capsules of miraculin 0.2 each (total 0.4 mg) were ingested 30 minutes before the meal was consumed.

Results are Shown for Subject 1

Subject 1--Falafel plus Two Purified Miraculin Capsules (0.2 mg each) total dose 0.4 mg
Symptom Severity (0-10)

| TIME | Abdominal | | Gas | Metallic | | |
|---|---|---|---|---|---|---|
| (minutes) | discomfort | Bloating | retention | taste | Reflux | Diarrhea |
| 0 | 0 | 0 | 0 | 0 | 0 | 0 |
| 15 | 0 | 0 | 2 | 0 | 0 | 0 |
| 30 | 1 | 1 | 0 | 0 | 0 | 0 |
| 45 | 1 | 0 | 1 | 0 | 0 | 0 |
| 60 | 0 | 0 | 0 | 0 | 3 | 0 |
| 75 | 0 | 0 | 0 | 0 | 3 | 0 |
| 90 | 0 | 0 | 0 | 0 | 2 | 0 |
| 105 | 0 | 0 | 0 | 0 | 4 | 0 |
| 120 | 0 | 0 | 0 | 0 | 4 | 0 |

Table 4-Study of dyspeptic subject after a falafel meal. Study consisted of the meal alone.

Results are Shown for Subject 2

Subject 2--Falafel Alone
Global Symptom Severity Score ranges from 0-10
Check marks indicate presence of specific symptoms

| | | | | | | | |
|---|---|---|---|---|---|---|---|
| TIME | Global | Abdominal | | Gas | Metallic | | |
| (minutes) | Score | discomfort | Bloating | retention | taste | Reflux | Diarrhea |

| | | | | | |
|---|---|---|---|---|---|
| 0 | 3 | ✓ | ✓ | ✓ | |
| 15 | 5 | ✓ | ✓ | ✓ | |
| 30 | 5 | ✓ | ✓ | ✓ | |
| 45 | 6 | ✓ | ✓ | ✓ | ✓ |
| 60 | 7 | ✓ | ✓ | ✓ | ✓ |
| 75 | 7 | ✓ | ✓ | ✓ | ✓ |
| 90 | 6 | ✓ | ✓ | ✓ | ✓ |
| 105 | 6 | ✓ | ✓ | ✓ | ✓ |
| 120 | 6 | ✓ | ✓ | ✓ | ✓ |

Table 5—Study of dyspeptic subject, two delayed release capsules of miracle berry extract were ingested 15 minutes before the meal was consumed. Results are shown for Subject 2

Subject 2--Falafel plus two Miracle Extract Berry Capsules
Global Symptom Severity Score ranges from 0-10
Check marks indicate presence of specific symptoms

| | | | | | | | |
|---|---|---|---|---|---|---|---|
| TIME | Global | Abdominal | | Gas | Metallic | | |
| (minutes) | Score | discomfort | Bloating | retention | taste | Reflux | Diarrhea |

| | | | | |
|---|---|---|---|---|
| 0 | 0 | | | |
| 15 | 1 | ✓ | | |
| 30 | 1 | ✓ | | |
| 45 | 0 | | | |
| 60 | 1 | | ✓ | ✓ |
| 75 | 0 | | | |
| 90 | 0 | | | |
| 105 | 0 | | | |
| 120 | 0 | | | |

Table 6—Study of a dyspeptic subject, consumed one delayed release capsule of purified miraculin 0.2 mg ingested 15 minutes before the meal was consumed.

Results are Shown for Subject 2

Subject 2--Falafel plus one purified miraculin 0.2 mg capsule
Global Symptom Severity Score ranges from 0-10
Check marks indicate presence of specific symptoms

| TIME | Global | Abdominal | | Gas | Metallic | | |
|---|---|---|---|---|---|---|---|
| (minutes) | Score | discomfort | Bloating | retention | taste | Reflux | Diarrhea |
| 0 | 0 | 0 | 0 | 0 | 0 | 0 | 0 |
| 15 | 1 | 2 | 1 | 2 | 0 | 1 | 0 |
| 30 | 1 | 2 | 1 | 1 | 0 | 0 | 0 |
| 45 | 2 | 2 | 2 | 0 | 0 | 1 | 0 |
| 60 | 0 | 0 | 0 | 0 | 0 | 0 | 0 |
| 75 | 1 | 2 | 1 | 0 | 0 | 1 | 0 |
| 90 | 1 | 1 | 0 | 0 | 0 | 2 | 0 |
| 105 | 1 | 2 | 0 | 0 | 0 | 0 | 0 |
| 120 | 2 | 2 | 0 | 0 | 0 | 3 | 0 |

Table 7—Dyspeptic subject, two delayed release capsules of purified miraculin 0.2 mg (total 0.4 mg) were ingested 15 minutes before the meal was consumed.

Results are Shown for Subject 2

Subject 2--Falafel plus two Miraculin Capsules (0.2 mg each), total 0.4 mg dose
Global Symptom Severity Score ranges from 0-10
Check marks indicate presence of specific symptoms

| TIME | Global | Abdominal | | Gas | Metallic | | |
|---|---|---|---|---|---|---|---|
| (minutes) | Score | discomfort | Bloating | retention | taste | Reflux | Diarrhea |
| 0 | 0 | 0 | 0 | 0 | 0 | 0 | 0 |
| 15 | 0 | 0 | 0 | 0 | 0 | 0 | 0 |
| 30 | 0 | 0 | 0 | 0 | 0 | 0 | 0 |
| 45 | 1 | 1 | 1 | 0 | 0 | 1 | 0 |
| 60 | 1 | 2 | 0 | 0 | 0 | 2 | 0 |
| 75 | 1 | 2 | 0 | 0 | 0 | 2 | 0 |
| 90 | 1 | 1 | 0 | 0 | 0 | 1 | 0 |
| 105 | 1 | 1 | 0 | 0 | 0 | 1 | 0 |
| 120 | 1 | 1 | 0 | 0 | 0 | 1 | 0 |

### DETAILED DESCRIPTION

Broadly, the present invention provides novel methods of treating a variety of symptoms related to functional GI disorders, including dyspepsia, food and medication intolerances and IBS. These methods may prevent the occurrence of symptoms of these disorders or reduce the severity or duration of these symptoms once present. The methods utilize a combination of the administration of the dried form, extract or chemically active protein of a naturally occurring fruit that contains a protein that binds to taste receptors. The dried fruit, extract or active protein is placed in a form such as a delayed release or pH-dependent capsule or tablet that prevents the destruction of the treatment in the acidic environment of the stomach, increases the retention time of the within the GI tract and promotes its release in the small or large intestine. Because the active components of these medications are proteins that bind rapidly to local taste receptors and would be destroyed in the acid environment of the stomach, their administration in a delayed release or acid protected form that allows them to have novel, previously undiscovered effects in the small and large intestine and to spread their effect over a large surface area of the GI tract.

In some embodiments the treatment may be administered as a delayed release capsule. In other embodiments, the treatment may be administered as a delayed release tablet. The delay in release of the treatment from the capsule or tablet may occur via a pH dependent property of the capsule or tablet. Alternatively, the delay in release of the capsule or tablet may be due to the physiologic or mechanical properties of the capsule or tablet, such as their acid resistance or slow dissolution. In some embodiments, the active ingredients may also be converted to microgranules that are contained in a standard release, or delayed release capsule or tablet. In some embodiments, the treatment may consist of dried fruit containing the treatment. Other forms may include an extract of the fruit, or a pharmaceutical grade of its active ingredient.

Miraculin is a glycoprotein that is found in the berries of the miracle berry plant (Synsepalum dulcificum). Both miracle berries and miraculin peptide in themselves have no specific taste. When miracle berries are chewed, they exhibit taste-modifying properties and convert the sensation of sour stimuli, such as lemon juice or vinegar to a sweet taste by their interaction with taste receptors on the tongue. Because the mechanism of action of miraculin peptide involves receptor modification, unlike the sweet sensation experienced for a short duration after exposure to sugars, the sensation of sweetness on the tongue and in the mouth continues for about one hour. Other natural proteins exist in nature with similar taste modifying effects on oral taste receptors.

The most commonly studied chemoreceptors that respond to sweet substances are found on the taste buds of the tongue and inside of the mouth. However, sweet receptors are known to occur throughout the body including the respiratory system, hypothalamus, testes, pancreatic islet cells, and the gastrointestinal tract. In the gastrointestinal tract, these sweet taste receptors are generally located on enteroendocrine cells.

At present, gastrointestinal sweet receptors have been postulated to have a role in the chemical analysis of luminal contents, and in the physiologic assessment of energy intake. Stimulation of intestinal sweet taste receptors causes the release of glucagon-like peptide 1 (GLP-1), which enhances glucose absorption and insulin secretion. Studies have shown that patients with type II diabetes have fewer gastrointestinal taste receptors and produce less GLP-1 after consuming a meal. Thus, it has been postulated that gastrointestinal sweet taste receptors may be involved with glucose absorption and insulin secretion in patients with Type II diabetes. Because of these properties, the present invention may have a future role in the management of diabetes. In addition, the present invention may have a role in modification of weight, either by promoting or decreasing food intake.

Based on our initial studies, we believe that gastrointestinal sweet taste receptors may play a role in improving sensory and motor disturbances seen in patients with functional gastrointestinal disorders. This role is in keeping with the location of these receptors on enteroendocrine cells. These cells are normally involved in regulatory activities of digestion and gastrointestinal motility. Research into the role of gastrointestinal receptor function and disorders such as functional dyspepsia and IBS has to date focused primarily on modifications occurring in the enteric nervous system. To this end, pharmacologic treatments for IBS have included serotonin 5-HT4 receptor agonists and 5-HT3 receptor agonists and activators and inhibitors of cholinergic, serotonergic, adrenergic, histaminergic, dopaminergic, gastrineric, nitric oxide, proton pumps and chloride transport receptors have been the focus of attention for pharmacologic intervention in functional GI disorders. Antagonists of gastrointestinal dopamine D2 receptors and serotonin 5-HT3 are commonly used for the treatment of post-operative and chemotherapy-induced nausea and vomiting. These agents are also commonly used for the treatment of functional dyspepsia, as are serotonin-reuptake inhibitors (SSRIs). These medications also have a role in the management of IBS. Although these pharmacologic agents have demonstrated clinical efficacy, they also have a number of potential serious side effects including those involving the cardiovascular and neurologic systems.

On the other hand, miracle berry and miraculin protein are regarded as safe food products. They have been considered for use as artificial sweeteners in the past, although they have not undergone regulatory approval for this use. Miracle berry is sold without prescription and is commonly used as a novelty food to produce unexpected sweet taste after the ingestion of sour foods. This effect occurs after chewing miracle berries. Chewing miraculin berries releases their active ingredient, miraculin protein, which then binds to cells containing the sweet receptors hT1R2-hT1R3 in the mouth. It is postulated that miraculin protein binds strongly to these receptors, thus producing receptor modification and neural signaling for prolonged periods of time. This property would make it likely that miraculin protein will have long lasting effects when binding to gastrointestinal sweet taste receptors as well, thus enhancing its pharmacologic efficacy.

Because the active ingredient of miracle berry is the glycoprotein miraculin, normal ingestion of miracle berries or miraculin is anticipated to only have some limited gastrointestinal effects, as miraculin will become denatured in the acidic environment of the stomach. The average intra-oral pH is approximately 7. This mildly alkaline environment is due to the secretion of sodium bicarbonate by the salivary glands. Since miraculin protein has been shown in animal studies to have effective and prolonged binding to sweet taste receptors in the mouth, it is anticipated that the neutral to alkaline environment of the small and large intestines are compatible with miraculin protein activity and represent a site of potential efficacy of miraculin protein.

In order to achieve this efficacy, miracle berry, whether as whole berry, dried juice, extract or as purified natural or synthetic miraculin will require administration in a form that will bypass the stomach to prevent the breakdown of the miraculin protein. Furthermore, efficacy will be enhanced if miracle berry and naturally derived or synthetic miraculin protein is administered in a form that will deliver high concentrations of its active ingredient to the site of action, in this case the small and large intestines.

The present invention uses a delivery system or systems that will achieve these desired effects by administering miracle berry, miracle berry juice, miracle berry extract or naturally derived or synthetic miraculin in a delayed release, sustained release, and/or microsphere forms. These may be either capsules or tablets. Other available and unique delivery forms of miracle berry and miraculin protein, as well as other fruits and their active proteins that bind to sweet taste receptors are also be included in this invention. Further modifications of these delivery systems will be made to optimize efficacy of miracle berry and miraculin protein. These modifications may involve development of unique delivery systems for these substances or use of existing delayed release or pH sensitive delivery systems to optimize the product. Administration of miracle berry, miracle berry juice and naturally derived or synthetic miraculin protein in delayed release or pH sensitive forms to produce binding of sweet taste receptors hT1R2-hT1R3 in the intestinal tract has not been previously proposed or published. In addition, this method or these substances have heretofore not been proposed as potential treatment of functional gastrointestinal disorders.

Delayed released substances that directly affect gastrointestinal neuroreceptors represent a potential important method for treating the symptoms of functional gastrointestinal disorders. This effect can be achieved using a variety of capsules that have release properties dependent on pH, capsule size or digestion by intestinal secretions. Furthermore, this affect can be achieved using pH-sensitive microparticles. This effect can also be achieved by developing coated microspheres of miracle berry extracts, dried miracle berry juice or naturally derived or synthetic miraculin peptide. Newly developed delayed release capsules and tablets may also be used to deliver miracle berry, miracle berry extract or miraculin protein to the small and large intestines.

Although aspects of the present invention herein have been described with reference to various embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

We claim:

1. A method for the treatment of functional dyspepsia, food intolerances, medication intolerances and irritable bowel syndrome comprising:
modifying intestinal sweet taste receptors.

2. The method of claim 1, wherein the step of modifying intestinal sweet taste receptors further includes the steps:
administering in an oral pharmacologic dose configured to include an active ingredient of at least one of the following: form of the whole miracle berry, dried miracle berry, miracle berry extract or purified miraculin.

3. The method of claim 2, wherein the oral pharmacologic dose is configured within one or more capsules or tablets for release in the small intestine and/or large intestine, thereby preventing the destruction of the active ingredients in the stomach.

4. The method of claim 3, wherein the capsule or tablet is a delayed release capsule configured to release the contents in the small intestine and/or large intestine.

5. The method of claim 3, wherein the active ingredient is delivered with a Ph-dependent delayed release capsule.

6. The method of claim 3, wherein the active ingredient is delivered with a slow release capsule.

7. The method of claim 3, wherein the active ingredient is further mixed with a substance configured to delay release of the active ingredient within the gastrointestinal tract.

8. The method of claim 2, wherein the active ingredient is delivered as microspheres, with or without a delayed release capsule that delays the release of the active ingredient within the gastrointestinal tract.

9. The method of claim 3, wherein the active dose of dried miracle berry is 1-10 dried miracle berries and the total dose for each usage will range from 1-5 capsules.

10. The method of claim 3, wherein the active dose of miracle berry is the juice of 1-30 miracle berries and the total dose for each usage will range from 1-5 capsules.

11. The method of claim 3, wherein the active dose of miracle berry extract is the extract from 1-30 miracle berries and the total dose for each usage will range from 1-5 capsules.

12. The method of claim 3, wherein the active dose of purified miraculin will range from 0.001 to 1 mg of miraculin protein and the total dose for each usage will range from 1-5 capsules.

13. The method of claim 8, wherein a dose of coated microspheres of miracle berry extract will range from the extract from 1-30 miracle berries and the total dose for each usage will range from 1-5 capsules.

14. The method of 1 further being configured for the management of diabetes.

15. The method of 1 further being configured for the management of weight disorders.

16. A delayed release capsule or tablet for the treatment of functional dyspepsia, food intolerances, medication intolerances and irritable bowel syndrome, comprising: a pharmacologic dose of having an active ingredient from at least one of the following:
form of the whole miracle berry, dried miracle berry, miracle berry extract or purified miraculin protein; or
sweet taste proteins including curculin, thaumatin, monellin, mabinlin, and pentadin and/or the natural fruits containing these sweet taste proteins.

17. The capsule or tablet of claim 16, wherein the active ingredient is miraculin protein, synthesized from miracle berry.

18. The capsule or tablet of claim 16, wherein the delayed release capsule or tablet is configured for release in the small intestine and/or large intestine, thereby preventing the destruction of the capsule or tablet in the stomach.

19. The capsule or tablet of claim 16, wherein the active ingredient is delivered with a Ph-dependent delayed release capsule.

20. The capsule or tablet of claim 16, wherein the active ingredient is delivered with a slow release capsule.

21. The capsule or tablet of claim 16, wherein the active ingredient is delivered mixed with a substance configured to delay release of the active ingredient within the gastrointestinal tract.

22. The capsule or tablet of claim 16, wherein active ingredient is delivered as microspheres, with or without a delayed release capsule that delays the release of the active ingredient within the gastrointestinal tract.

23. The capsule or tablet of claim 16, wherein the active dose of dried miracle berry is 1-10 dried miracle berries and the total dose for each usage will range from 1-5 capsules.

24. The capsule or tablet of claim 16, wherein the active dose of miracle berry is the juice of 1-30 miracle berries and the total dose for each usage will range from 1-5 capsules.

25. The capsule or tablet of claim 16, wherein the active dose of miracle berry extract is the extract from 1-30 miracle berries and the total dose for each usage will range from 1-5 capsules.

26. The capsule or tablet of claim 16, wherein the active dose of purified miraculin will range from 0.001 to 1 mg of miraculin protein and the total dose for each usage will range from 1-5 capsules.

27. The capsule or tablet of claim 16, wherein a dose of coated microspheres of miracle berry extract will range from the extract from 1-30 miracle berries and the total dose for each usage will range from 1-5 capsules.

28. The capsule or tablet of claim 16 further being configured for the management of diabetes or for the management of weight disorders.
